(19) United States
(12) Patent Application Publication
(10) Pub. No.: US 2025/0272457 A1
(43) Pub. Date: Aug. 28, 2025

(54) INFORMATION PROCESSING SYSTEM, INFORMATION PROCESSING METHOD, RECORDING MEDIUM

(71) Applicant: CANON KABUSHIKI KAISHA, Tokyo (JP)

(72) Inventor: TAKUJI OISHI, Kanagawa (JP)

(21) Appl. No.: 19/063,964

(22) Filed: Feb. 26, 2025

(30) Foreign Application Priority Data
Feb. 28, 2024 (JP) 2024-029114

(51) Int. Cl. G06F 30/27

(52) U.S. Cl. CPC G06F 30/27

(57) ABSTRACT

An information processing method performed by an information processing unit includes obtaining descriptors related to a material, converting the obtained descriptors into correlativity-reduced descriptors in which correlativity therebetween is reduced by using conversion coefficients, calculating importance of the correlativity-reduced descriptors, identifying important descriptors from the descriptors on a basis of the conversion coefficients used for conversion into the correlativity-reduced descriptors having high importance, generating a learned model by machine learning using the important descriptors and a characteristic value of the material as learning data, and estimating a characteristic value of a different material different from the material by using the learned model.

### BACKGROUND

### Technical Field

The present disclosure relates to an information processing system and the like used for estimating a physical property of a material of a resulting product from material compositions and processing conditions.

### Description of the Related Art

A technique of materials informatics (MI) in which a learned mode (physical property estimation model) is generated by performing machine learning of data of trial production conditions such as material compositions and processing conditions and data of material physical properties (evaluation value) of a resulting product and the material physical properties of a product that has never been produced for trial are estimated by using the learned model is known. In material development using MI, the physical properties of a material obtained in a trial production condition that has never been used are estimated. A trial production condition with which desired material physical properties are estimated to be obtained is identified by using this result. Then, a trial product of the material is actually produced by using the identified trial production condition, and whether or not the desired material physical properties are obtained is checked. At this time, if the precision of the estimation by the physical property estimation model is low, the precision of the identification of the trial production condition is also low, and the material development cannot be performed efficiently.

The learning data used for generating the physical property estimation model includes explanatory variables and a response variable. In material development, the explanatory variables indicate trial production conditions such as the amount and characteristics of the raw material to be used and processing conditions, and the response variable indicates an evaluation value of the characteristics of the developed material and the like. In some cases, descriptors that are values obtained by conversion according to the molecular structure, characteristics, and the like are used to improve the precision of the estimation model. Hundreds to thousands of descriptors can be generated from one piece of molecular information by using calculation, a database, or the like, and the number of the explanatory variables increases according to this. In the case of obtaining the learning data that is a set of explanatory variables and a response variable, the number of data sets that can be obtained is limited due to practical restrictions. In such a case, the learning data is data with a large number of explanatory variables and a small number of data sets, and it is difficult to improve the precision of the physical property estimation model therewith.

Explanatory variables are selected to improve the precision of the physical property estimation model. In this case, among the large number of explanatory variables, ones related to the response variable need to be kept and ones not related to the response variable need to be deleted. Relevant descriptors can be selected if the mechanism of a phenomenon is known, but the explanatory variables are selected by analyzing data if such knowledge is not available.

In Japanese Patent Application Laid-Open No. 2021-174403, the importance of the explanatory variables is estimated from the data by using Permutation Importance or the like, and explanatory variables of high importance are selected. In addition, in Japanese Patent Application Laid-Open No. 2022-108269, the explanatory variables are converted by using principal component analysis.

However, in the method described in Japanese Patent Application Laid-Open No. 2021-174403, although Permutation Importance or the like is used, the importance cannot be accurately evaluated and appropriate selection cannot be made in the case where the explanatory variables are correlated with each other.

In addition, in the method described in Japanese Patent Application Laid-Open No. 2022-108269, although the correlation between the explanatory variables can be reduced by principal component analysis, since the principal component is a value that does not have a physical meaning, important explanatory variables cannot be selected. Therefore, in material development in which not many sets of data can be obtained, an estimation model of high precision cannot be obtained, and the material development cannot be performed efficiently.

Therefore, in the field of material development, an information processing system capable of appropriately identifying important explanatory variables and estimating the material physical properties with high precision even in the case where not a lot of learning data is available has been desired.

### SUMMARY

According to a first aspect of the present disclosure, an information processing method performed by an information processing unit includes obtaining descriptors related to a material, converting the obtained descriptors into correlativity-reduced descriptors in which correlativity therebetween is reduced by using conversion coefficients, calculating importance of the correlativity-reduced descriptors, identifying important descriptors from the descriptors on a basis of the conversion coefficients used for conversion into the correlativity-reduced descriptors having high importance, generating a learned model by machine learning using the important descriptors and a characteristic value of the material as learning data, and estimating a characteristic value of a different material different from the material by using the learned model.

According to a second aspect of the present disclosure, an information processing system comprises an information processing unit. The information processing unit is configured to obtain descriptors related to a material, convert the obtained descriptors into correlativity-reduced descriptors in which correlativity therebetween is reduced by using conversion coefficients, calculate importance of the correlativity-reduced descriptors, identify important descriptors from the descriptors on a basis of the conversion coefficients used for conversion into the correlativity-reduced descriptors having high importance, generate a learned model by machine learning using the important descriptors and a characteristic value of the material as learning data, and estimate a characteristic value of a different material different from the material by using the learned model.

Further features of the present disclosure will become apparent from the following description of exemplary embodiments with reference to the attached drawings.

### BRIEF DESCRIPTION OF THE DRAWINGS

FIG. 1 is a schematic functional block diagram illustrating a configuration of functional blocks of an information processing system according to an embodiment.

FIG. 2 is a diagram illustrating an example of learning data obtained in processing of step S1.

FIG. 3 is a diagram illustrating an example of data obtained in processing of step S2.

FIG. 4 is a diagram illustrating an example of data obtained in processing of step S3.

FIG. 5 is a diagram illustrating an example of importance of each correlativity-reduced component obtained in processing of step S4.

FIG. 6 is a diagram illustrating an example of conversion coefficients obtained in processing of step S5.

FIG. 7 is a diagram illustrating an example of learning data constituted by a response variable and important explanatory variables extracted in processing of step S6.

FIG. 8 is a diagram illustrating an example of a relationship between explanatory variables and a weighted average of conversion coefficients obtained in a second embodiment.

FIG. 9 is a flowchart illustrating an information processing procedure according to an embodiment.

FIG. 10 is a schematic diagram illustrating an example of a hardware configuration of an information processing system according to an embodiment.

### DESCRIPTION OF THE EMBODIMENTS

An information processing system and the like that are embodiments of the present disclosure will be described with reference to drawings. To be noted, the embodiments shown below are merely examples, and for example, details of configurations thereof may be appropriately modified by one skilled in the art for implementation within the gist of the present disclosure.

To be noted, in the description of embodiments and examples below, it is assumed that elements denoted by the same reference signs have substantially the same functions unless otherwise described. In the drawings, in the case where a plurality of the same elements are provided, addition of reference signs and description thereof may be omitted in some cases. In addition, since the illustration may be schematically expressed in some cases for the sake of convenience of illustration and description, the shapes, sizes, layouts, and the like of the elements illustrated in the drawings do not necessarily strictly match those in the reality.

### First Embodiment

### Configuration of Functional Blocks

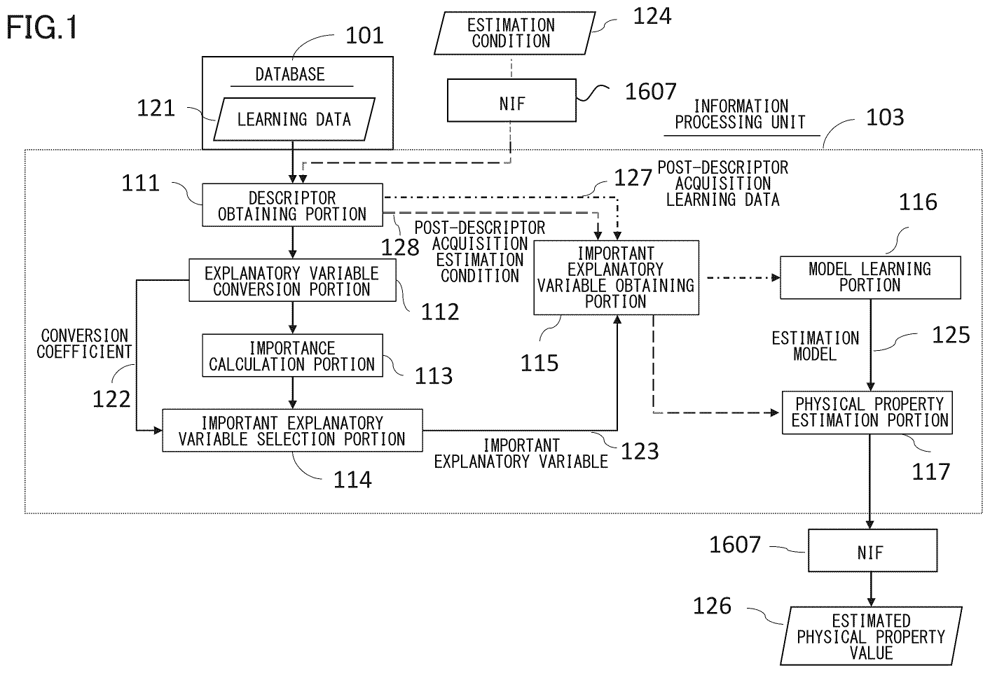

FIG. 1 is a schematic functional block diagram illustrating a configuration of functional blocks of an information processing system according to an embodiment. To be noted, in FIG. 1, functional elements required for describing the characteristics of the present embodiment are expressed by functional blocks, and description of general functional elements not directly related to the problem-solving principle of the present disclosure is omitted. In addition, each functional element illustrated in FIG. 1 is functionally conceptual, and does not need to be physically structured as illustrated. For example, the specific form of division and integration of the functional blocks is not limited to the illustrated example, and the entirety or part thereof may be functionally or physically divided and integrated in arbitrary units in accordance with the usage situation or the like.

Each functional block can be constituted by hardware or software. These functional blocks can be constituted by, for example, a central processing unit (CPU) reading a control program stored in a storage device or a non-transitory recording medium. Alternatively, the entirety or part of the functional blocks may be constituted by hardware such as an application-specific integrated circuit (ASIC) included in the information processing system.

An information processing unit 103 of the information processing system of the embodiment includes a descriptor obtaining portion 111, an explanatory variable conversion portion 112, an importance calculation portion 113, an important explanatory variable selection portion 114, an important explanatory variable obtaining portion 115, a model learning portion 116, and a physical property estimation portion 117. The function of each portion will be described later in association with a procedure of processing of estimating the material physical properties. The information processing unit 103 can obtain learning data from a database 101 storing learning data 121, obtain an estimation condition 124 from the outside via a network interface 1607, and output an estimated physical property value to the outside via the network interface 1607.

### Configuration of Hardware

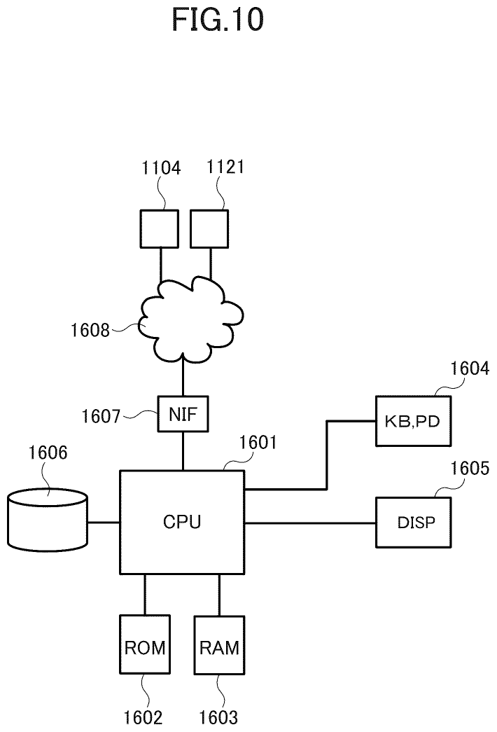

A schematic example of a hardware configuration of the information processing system according to the present embodiment will be described with reference to FIG. 10. The information processing system according to the present embodiment may be a physical calculator system, or a system established on a calculation resource group such as a cloud structure.

As illustrated in FIG. 10, the information processing system can include personal computer (PC) hardware including a CPU 1601 serving as a main control device, a read-only memory (ROM) 1602 serving as a storage device, and a random-access memory (RAM) 1603. The ROM 1602 can store information such as a processing program for executing an information processing method that will be described later. In addition, the RAM 1603 is used as a work area for the CPU 1601 when executing the information processing method. In addition, the PC hardware is connected to an external storage device 1606. The external storage device 1606 is constituted by a hard disk drive (HDD), a solid-state device (SSD), an external storage device of a different system that is network-mounted, or the like.

A processing program of the CPU 1601 for realizing the information processing apparatus or the information processing method according to the embodiment can be stored in a storage portion such as the external storage device 1606 constituted by an HDD, an SSD, or the like, or the ROM 1602 (for example, in an electrically erasable programmable ROM (EEPROM) area). In this case, the processing program of the CPU 1601 for realizing the information processing method (method for estimating the material physical properties) can be supplied to each storage portion described above or updated to a new different program via the network interface 1607. Alternatively, the processing program of the CPU 1601 for realizing the information processing method can be supplied to each storage portion described above or updated to a new different program via storage means such as various magnetic disks, optical disks, and flash memories and drive devices therefor. Various storage means, storage portions, and storage devices storing a program with which processing of the CPU 1601 for realizing the information processing method serve as a computer-readable recording medium for the information processing method or the information processing apparatus of the present disclosure.

The network interface 1607 can be constituted by, for example, using a communication standard for wired communication such as IEEE 802.3, or wireless communication such as IEEE 802.11 or IEEE 802. 15. The CPU 1601 can communicate with external apparatus 1104 and 1121 on a cloud 1608 via the network interface 1607. For example, the external apparatus 1104 and the external apparatus 1121 each may be an integral control apparatus or a management server such as a programmable logic controller (PLC) or a sequencer provided for controlling or managing a material trial production apparatus or a material analysis apparatus.

In an example illustrated in FIG. 10, an operation portion 1604 serving as an input portion and a display apparatus 1605 serving as an output portion are connected as a user interface apparatus (UI apparatus). The operation portion 1604 can be constituted by a terminal such as a handy terminal, or a device such as a keyboard, a jog dial, a mouse, a pointing device, a sound input device, or the like (or a control terminal including these). The display apparatus 1605 may be any device as long as the display apparatus 1605 can display, on a display screen, information related to processing executed by the important explanatory variable selection portion 114, the important explanatory variable obtaining portion 115, the physical property estimation portion 117, and the like that will be described later with reference to FIG. 1, and for example, a liquid crystal display apparatus can be used.

To be noted, the hardware configuration of the information processing system according to the embodiment is not limited to the example of FIG. 10, and may include a processor (for example, a graphics processing unit) suitable for generating a learned model by machine learning.

### Information Processing Procedure

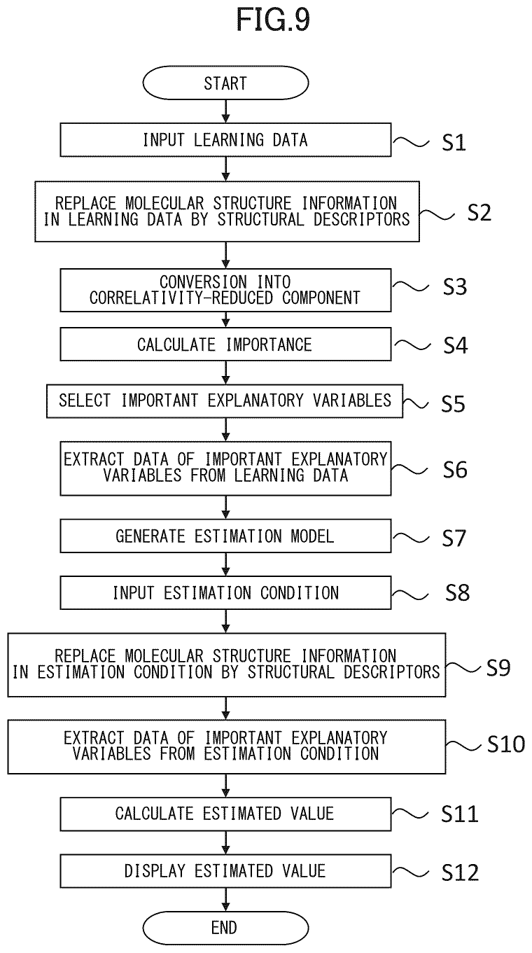

A information processing procedure related to estimation of material physical properties will be described in association with the function of each portion of the information processing unit 103 (FIG. 1). FIG. 9 illustrates a flowchart indicating the information processing procedure.

When the processing is started, in step S1, the information processing unit 103 obtains the learning data 121 stored in the database 101. The learning data 121 may be read from the database 101 provided on the inside or the outside, or may be input from the outside via the network interface 1607.

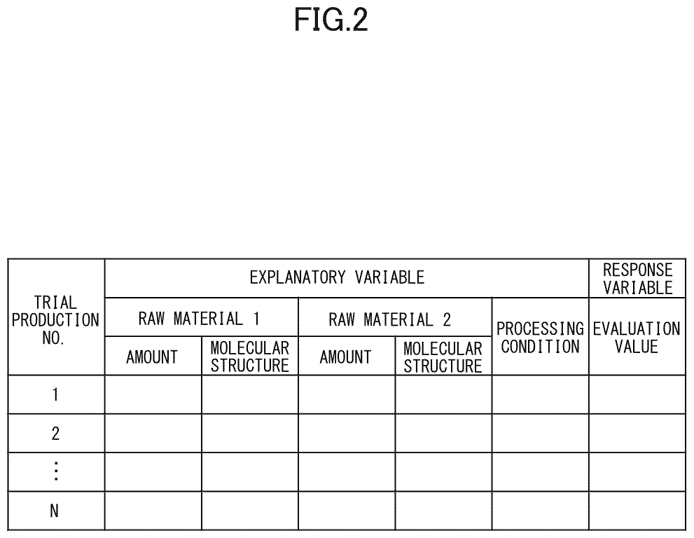

The learning data 121 will be described with reference to an example illustrated in FIG. 2. The learning data 121 includes sets of explanatory variables and a response variable by the number of times of trial production. The explanatory variables include information such as the use amount, molecular structure, physical property values, processing conditions, and the like of the raw material. Although the molecular structure is preferably expressed in the SMILES format for easier calculation, the format is not limited to this as long as the molecular structure is accurately expressed. The response variable includes information of a physical property value, that is, information of an evaluation value of the material obtained by the trial production. The response variable may include information of one evaluation value or a plurality of evaluation values.

Next, in step S2, the descriptor obtaining portion 111 of the information processing unit 103 obtains descriptors from the information of the molecular structure included in the learning data 121 obtained in step S1, and replaces the item of the molecular structure in the learning data 121 by descriptors (for example, structural descriptors).

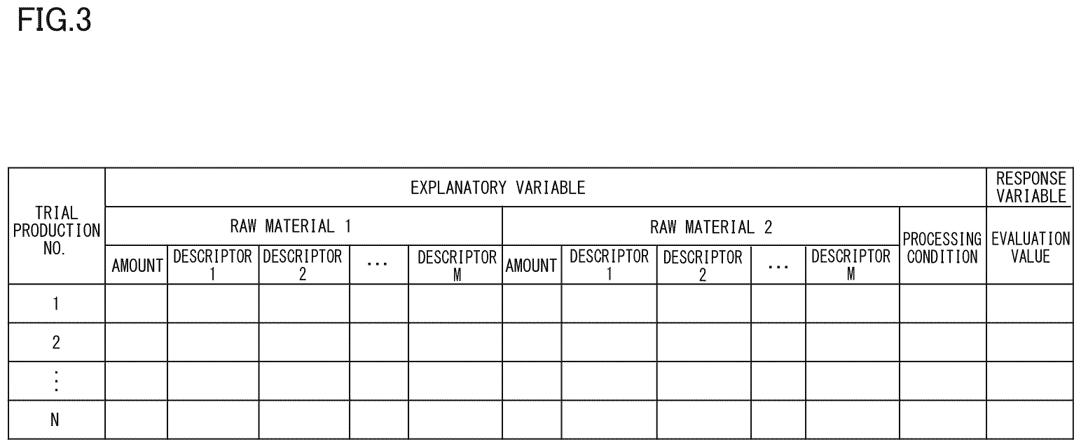

FIG. 3 illustrates an example of data obtained by this processing. Here, N represents the number of data sets, and M represents the number of descriptors. As a result of this processing, the number of explanatory variables increases, and M becomes hundreds to thousands. At this time, the effect of the implementation of the present disclosure is especially likely to be obtained in the case where the relationship between the number N of the data sets and the number P of the explanatory variables (P=2M+3 in the example of FIG. 3) is N<P. In addition, the present disclosure can be implemented even in the case of N≥P.

The descriptors may be calculated from the molecular structure, or may be obtained by referring to a database including molecular information and descriptors. In the case of obtaining the descriptors by calculation, an open-source library such as RDKit or Mordred may be used. The obtained descriptors are preferably related to the evaluation value of the response variable, but may be all descriptors that can be obtained. At this time, the user may select arbitrary descriptors by using an unillustrated input device. The explanatory variables including the descriptors obtained herein include ones correlated with each other, and the importance of the explanatory variables cannot be correctly evaluated in this state.

Therefore, in step S3, the explanatory variable conversion portion 112 of the information processing unit 103 performs processing of reducing the correlativity, that is, replaces the explanatory variables by a component in which the correlativity between the explanatory variables is reduced, in other words, by a correlativity-reduced component.

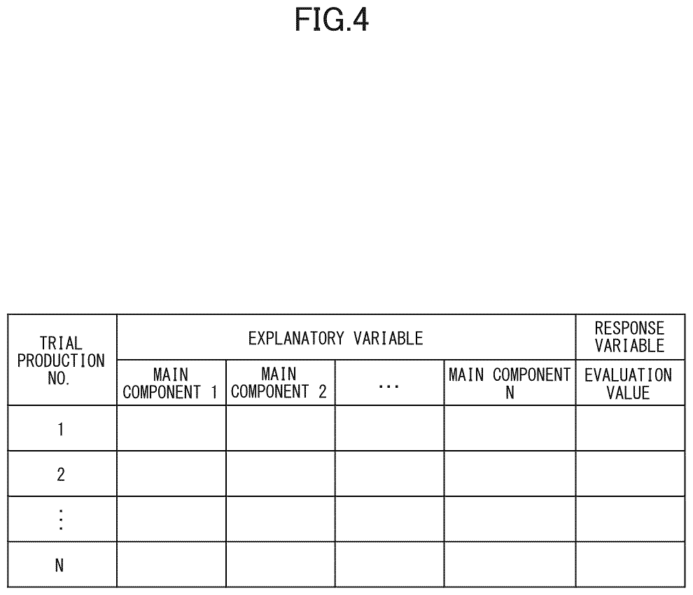

FIG. 4 illustrates an example of the learning data obtained by this processing. It is preferable that the explanatory variables of the learning data 121 obtained by the descriptor obtaining portion 111 are standardized, that is, scaled such that the average thereof is 0 and the standard deviation thereof is 1. The explanatory variables subjected to this processing are treated as standardized explanatory variables. As a result of this, important explanatory variables (important descriptors) can be appropriately selected even in the case where the numerical range of the explanatory variables differs between the explanatory variables. To be noted, the standardization does not need to be performed in the case where the numerical range of the explanatory variables is close between the explanatory variables. The explanatory variables are treated as the standardized explanatory variables after this step even in the case where the standardization is not performed.

Next, by performing principal component analysis (PCA) by using the standardized explanatory variables, a unique vector is obtained, and this unique vector is used as a conversion coefficient A. Singular value decomposition (SVD) may be used instead of principal component analysis. Next, conversion using the following formula (1) is performed by using the conversion coefficient A.

$$E = AD \qquad Formula ( 1 )$$

Here, E, A, and D are respectively a matrix of the correlativity-reduced component, a conversion matrix, and a matrix of the standardized explanatory variables. The formula (1) can be expressed as the following formula (2) by spreading out the elements thereof.

$$[ \begin{matrix}\begin{matrix}{E}_{1} \\ ⋮\end{matrix} \\ E \text{?}\end{matrix} ] = [ \begin{matrix}{A}_{11} & … & A \text{?} \\ ⋮ & ⋱ & ⋮ \\ A \text{?} & … & A \text{?}\end{matrix} ] [ \begin{matrix}\begin{matrix}{D}_{1} \\ ⋮\end{matrix} \\ D \text{?}\end{matrix} ] \qquad Formula ( 2 )$$

$$\text{?} \text{indicates text missing or illegible when filed}$$

Here, Eᵢ represents the i-th correlativity-reduced component, Dⱼ represents the j-th standardized explanatory variable, Aᵢⱼ represents a coefficient of Dⱼ for calculating Eᵢ. By performing the conversion of the formula (2) on one set of trial production data, that is, on one row of FIG. 3, a corresponding row in FIG. 4 can be obtained. Therefore, this conversion process is sequentially performed for all trial productions. By performing this conversion, the correlativity-reduced components are orthogonalized with respect to each other, and correlativity-reduced descriptors that are explanatory variables from which correlativity has been reduced can be obtained.

Next, in step S4, the importance calculation portion 113 of the information processing unit 103 obtains the importance of the explanatory variables. Specifically, the importance is calculated by analyzing an estimation model generated by using the learning data in which the explanatory variables have been replaced by the correlativity-reduced descriptors by the explanatory variable conversion portion 112.

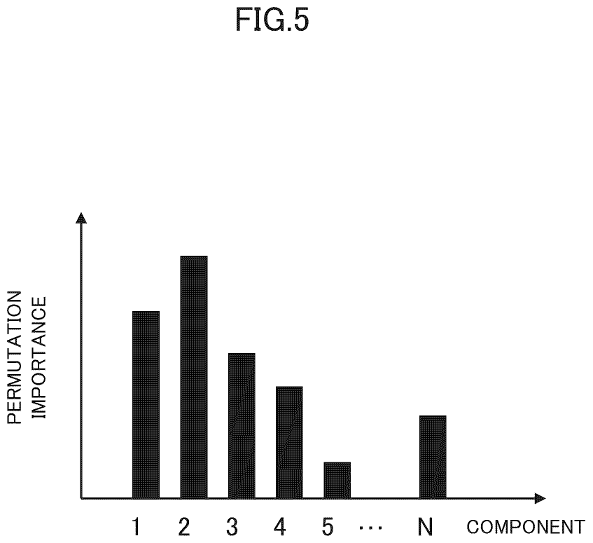

FIG. 5 illustrates an example of importance of each correlativity-reduced component calculated by this processing. It is preferable that Permutation Importance is used for calculation of the importance. Permutation Importance is a method in which the performance of an estimation model (learned model) generated by using the learning data is evaluated in advance, the performance of an estimation model generated by swapping certain explanatory variables in the learning data is evaluated, and the importance of the explanatory variables is evaluated by evaluating the decrease in the performance. Examples of the algorithm of machine learning that can be used at this time include Ridge regression, support vector regression, random forest, neural network, and gradient boosting decision tree. Alternatively, a different algorithm for machine learning in which the relationship between the explanatory variables and the response variable may be used. In addition, the estimation model (learned model) may be generated by using a plurality of algorithms, the performance of the estimation model may be evaluated by a method such as cross-validation, and the algorithm with the best performance may be employed. In addition, after evaluating the performance of the estimation model, the user may select the algorithm by referring to the evaluation results. In addition, in the case of calculating the importance by using an algorithm of a decision tree type, Feature Importance by the decision tree may be obtained, and in the case of calculating the importance by using an algorithm of Lasso regression, a coefficient of linear combination may be used as the importance.

Next, in step S5, the important explanatory variable selection portion 114 of the information processing unit 103 selects important explanatory variables as important descriptors from the explanatory variables. Although the importance of each correlativity-reduced component is obtained by the processing of step S4, each correlativity-reduced component does not have physical meaning. Therefore, important explanatory variables cannot be selected in this state. In contrast, the relationship between the explanatory variables and the correlativity-reduced component is described in conversion coefficients used in the processing of the explanatory variable conversion portion 112. Therefore, by investigating a conversion coefficient 122 related to the important correlativity-reduced component, the correlativity-reduced component can be converted back to explanatory variables, and important explanatory variables (important descriptors) can be known.

The important explanatory variable selection portion 114 selects the important explanatory variables by using the method described above. Specifically, first, the most important correlativity-reduced component E_{M} is selected on the basis of the processing result of step S4. Next, conversion coefficients A_{MI} to A_{MP} used for obtaining the most important correlativity-reduced component E_{M} in the conversion performed by the explanatory variable conversion portion 112 are referred to. These conversion coefficients can be positive or negative values, so absolute values thereof are obtained to compare the magnitude.

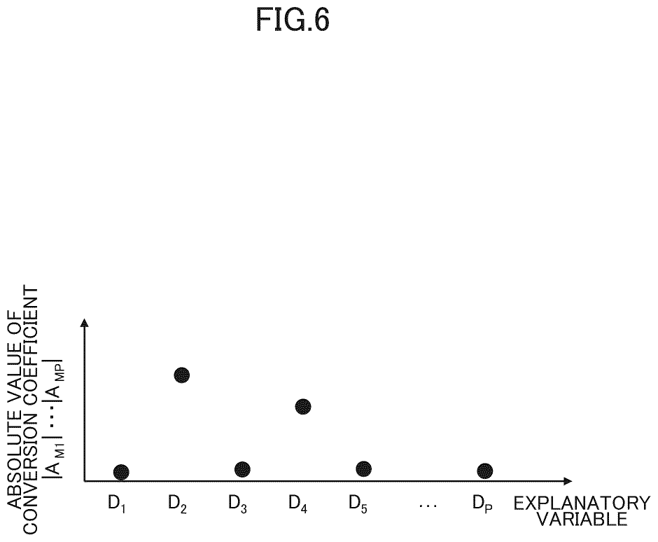

FIG. 6 illustrates an example of the obtained conversion coefficients |A_{MI}| to |A_{MP}|. Explanatory variables corresponding to conversion coefficients having a large absolute value are selected as important explanatory variables 123. To be noted, the conversion coefficients A_{MI} to A_{MP} are coefficients of the explanatory variables D₁ to D_{P}, and therefore these values correspond to each other.

As illustrated in FIG. 6, normally, there are more than one important explanatory variable (and conversion coefficient corresponding thereto). Four methods to select conversion coefficients of large values from the obtained conversion coefficients |A_{MI}| to |A_{MP}| will be described below.

(1) The number of conversion coefficients (predetermined number) to be selected may be determined in advance, and descriptors may be selected in the order of larger values. According to this, an appropriate number of explanatory variables can be selected in consideration of the number of data sets to be used, and thus an estimation model of high precision can be obtained. For example, the number of the explanatory variables to be selected is preferably about 0.1N to 0.5N with respect to the number N of data sets, but the number may be deviated from this value depending on the case.

(2) In addition, the maximum value of |A_{MI}| to |A_{MP}| may be standardized to 1, and values larger than a predetermined threshold value determined in advance may be selected. In the method (1), in the case where the number of truly important explanatory variables is smaller than the set number of explanatory variables, unnecessary explanatory variables are selected, and thus the precision of the estimation model deteriorates. Such a situation can be avoided with the method (2).

(3) In addition, the methods (1) and (2) are preferably combined. That is, the number of conversion coefficients to be selected is determined in advance, the maximum value of |A_{MI}| to |A_{MP}| is standardized to 1 and a threshold value thereof is determined in advance, and only explanatory variables satisfying both conditions are selected. The method (1) has the problem described above, and in the method (2), the number of explanatory variables to be selected with respect to the number of data sets can be excessive, and there is a possibility that the precision of the estimation model cannot be improved. In the method (3), both problems can be avoided, and thus an estimation model of high precision can be obtained.

(4) As illustrated in FIG. 6, the values of | A_{MI}| to |A_{MP}| may be displayed on the display apparatus 1605, and the user may manually select the explanatory variables. In this case, the obtained result depends on the user, but can flexibly adapt to the situation.

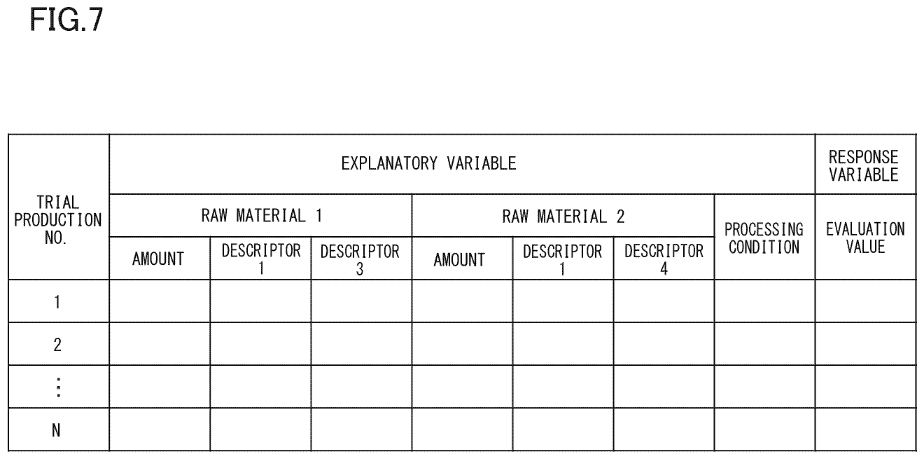

Next, in step S6, the important explanatory variable obtaining portion 115 of the information processing unit 103 extracts only data (characteristic values of the material) of explanatory variables matching the selected important explanatory variables 123 from post-descriptor acquisition learning data 127 output from the descriptor obtaining portion 111. FIG. 7 illustrates an example of learning data constituted by the extracted explanatory variables and the response variable.

Next, in step S7, the model learning portion 116 of the information processing unit 103 generates an estimation model 125 (learned model) by using the learning data constituted by the important explanatory variables and the response variable. For the generation of the estimation model 125, an algorithm of machine learning is used. As the algorithm, similarly to calculation of Permutation Importance, algorithms such as Ridge regression, support vector regression, random forest, neural network, and gradient boosting regression tree can be used. Alternatively, a different algorithm for machine learning that can express the relationship between the explanatory variables and the response variable may be used. In addition, the estimation model may be generated by using a plurality of algorithms, the performance of the estimation model may be evaluated by a method such as cross-validation, and the algorithm with the best performance may be employed. In addition, after evaluating the performance of the estimation model, the user may select the algorithm by referring to the evaluation results. The generated estimation model 125 is configured by the physical property estimation portion 117 as a learned model that can be executed.

Next, in step S8, an estimation condition 124 is input to the physical property estimation portion 117 by the user via the network interface 1607 or an unillustrated input portion. The estimation condition 124 is constituted by the same explanatory variables as the learning data 121, and the response variable is not needed. At this time, not only one but a plurality of conditions may be input. To be noted, the processing of step S8 may be performed subsequently to the step S1, and in this manner, the user can perform the input work collectively.

Next, in step S9, the descriptor obtaining portion 111 obtains descriptors from the molecular structure information of the estimation condition, and replaces the descriptors by structural descriptors.

Next, in step S10, the important explanatory variable obtaining portion 115 extracts descriptors matching the important explanatory variables from the estimation condition. To be noted, to simplify the processing, the descriptor obtaining portion 111 may obtain only descriptors matching the important explanatory variables instead of steps S9 and S10.

Next, in step S11, when the estimation condition from which only the important explanatory variables have been extracted is input, an estimated physical property value 126 (characteristic value of a material different from the material used for the learning data) is output by the estimation model 125 configured by the physical property estimation portion 117. To be noted, although the estimation condition is the same as the explanatory variables of the learning data 121, a post-descriptor acquisition estimation condition 128 or an estimation condition obtained by extracting only the important explanatory variables may be directly input. In addition, although the estimation condition may be input directly, when obtaining a plurality of conditions, a generation rule may be set, and the plurality of conditions may be automatically generated in accordance with the rule.

Next, in step S12, the estimated physical property value 126 serving as a calculation result is displayed on the display apparatus 1605 (FIG. 10). The estimated physical property value 126 may be output to the outside via the network interface 1607.

In the present embodiment, an information processing system (material physical property estimation model) capable of estimating a physical property value with high precision even in the case where there is not a lot of learning data by obtaining the estimated physical property value by using only the important explanatory variables in this manner can be obtained.

### Second Embodiment

An information processing system according to a second embodiment will be described. Description of elements common to the first embodiment will be simplified or omitted.

In the first embodiment, the most important correlativity-reduced component E_{M} is selected by the important explanatory variable selection portion 114 from the result of the importance calculation portion 113, but the information for selecting the important explanatory variables included in the correlativity-reduced components not having the highest importance is not used. In the second embodiment, this information is also used to select the important explanatory variables more appropriately, and is different from the first embodiment in this point. Two forms of the present embodiment will be described below.

(1) The important explanatory variable selection portion 114 selects a predetermined number T from the correlativity-reduced components E₁ to E_{N} output from the importance calculation portion 113 in the order of high importance. Absolute values |A_{S11}| . . . |A_{SIP}| to |A_{STI}| . . . |A_{STP}| of the conversion coefficients for obtaining the selected correlativity-reduced components E_{S1} to E_{ST} are referred to, and explanatory variables of large values are selected similarly to the first embodiment. At this time, explanatory variables can be redundantly selected, and therefore redundant explanatory variables are removed and important explanatory variables are obtained. Alternatively, redundant ones may be regarded as more important, and the explanatory variables may be further narrowed down on the basis of the number of times the explanatory variables have been selected.

(2) In the important explanatory variable selection portion 114, important explanatory variables (important descriptors) are selected on the basis of a weighted average value of the absolute values of the conversion coefficients Aᵢⱼ obtained by the explanatory variable conversion portion 112 by using the importance of each correlativity-reduced component output from the importance calculation portion 113 as the weight. Specifically, the weighted average value Sᵢ corresponding to each explanatory variable Dᵢ is calculated by using the following formula (3).

$${S}_{i} = \frac{\sum \text{?} \vert A \text{?} \vert \times E \text{?}}{\sum \text{?} E \text{?}} \qquad Formula ( 3 )$$

$$\text{?} \text{indicates text missing or illegible when filed}$$

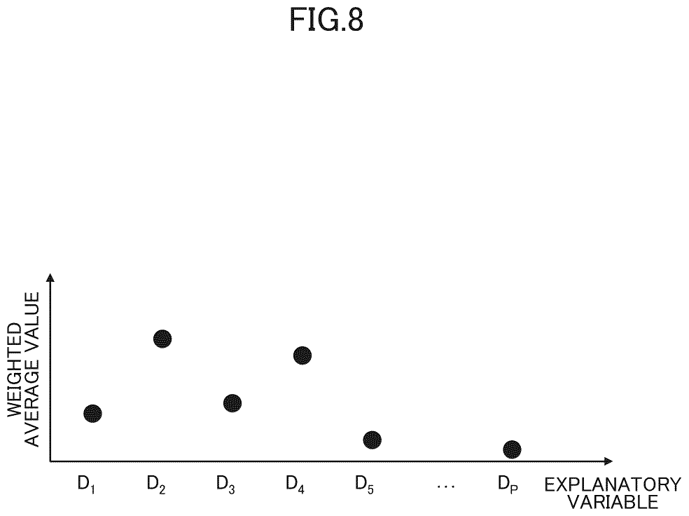

FIG. 8 illustrates an example of results obtained by this calculation. The explanatory variable Dᵢ for which the weighted average value Sᵢ is large is selected. The method of selection is the same as the method of selecting conversion coefficients of large values from the conversion coefficients |A_{MI}| to |A_{MP}| of the first embodiment.

In the second embodiment, since the selection of the explanatory variables is performed more appropriately, an information processing system (material physical property estimation model) capable of estimating the physical property value with higher precision even if there is not a lot of learning data can be obtained.

### EXAMPLE

An example in which the information processing system according to the second embodiment was applied to material development for realizing a material having a high elastic modulus by mixing aromatic amine with bismaleimide and curing the mixture will be described.

First, learning data constituted by trial production conditions and evaluation values was generated by evaluating the elastic modulus while changing the kind of materials and mixture amount of bismaleimide and aromatic amine. Only one kind of bismaleimide (BMI-70 manufactured by K⋅I CHEMICAL INDUSTRY CO., LTD.) was used in this case. In addition, five kinds of aromatic amine (WANAMINE MDA-100H, MDA-100, ETHACURE 100+, ETHACURE 300, and 4,4′-diaminodiphenyl sulfone manufactured by MITSUI FINE CHEMICALS, INC.) were used. The number of obtained data sets was 25. The explanatory variables of the learning data expressed the proportion of the amount of the aromatic amine with respect to the amount of bismaleimide and the molecular structure of each material of the aromatic amine in the SMILES format.

A file in which this learning data was described was input to the information processing system according to the second embodiment, and a material physical property estimation model (learned model) was obtained. The following processing was performed in the information processing system.

400 descriptors related to the molecular structure was obtained from the molecular structure information of each material in the SMILES format by using RDKit. As a result of this, 401 explanatory variables including the proportion of the amount of aromatic amine with respect to the amount of bismaleimide and the descriptors of the aromatic amine were obtained. Principal Component Analysis was performed on this data, and a correlativity-reduced component was obtained. At this time, the number of explanatory variables was reduced to 25.

The material physical property estimation model was generated for this data by using several machine learning algorithms. At this time, cross-validation was performed, and the material physical estimation model was selected by using a coefficient of determination as an index. As a result of this, a method of using Light GBM of gradient boosting was the most precise, and the coefficient of determination thereof was 0.81. As a result of calculating the Permutation Importance by using that material physical estimation model, the 3 correlativity-reduced components with highest importance were a main component 3, a main component 15, and a main component 5.

The conversion coefficient of each corresponding main component was referred to, and 3 explanatory variables of the largest absolute values each were selected, and thus 9 explanatory variables were selected in total. The selected explanatory variables were reduced to 5 by removing redundant explanatory variables, and the 5 were the proportion of the amount of aromatic amine with respect to the amount of bismaleimide, MolWt of the aromatic amine, TPSA of the aromatic amine, BertzCT of the aromatic amine, and SMR_VSA7 of the aromatic amine.

The explanatory variables described above were extracted from the learning data subjected to descriptor conversion, estimation models were generated by several machine learning algorithms, and the most precise model was employed as the estimation model by performing cross-validation using the coefficient of determination as an index. The coefficient of determination of cross-validation of this estimation model was 0.92, which indicated high precision.

As estimation conditions, 18 conditions were set by combining 6 conditions in which the proportion of the amount of aromatic amine with respect to the amount of bismaleimide was respectively 0.05, 0.1, 0.15, 0.2, 0.25, and 0.3 and 3 kinds of aromatic amine including bisaniline-M, bisaniline-P, and ETHACURE (registered trademark) 100+ manufactured by MITSUI FINE CHEMICALS, INC.). The data format at this time was set to be the same as that of the explanatory variables of the learning data. A file describing these estimation conditions was input to the information processing system, and an estimation value of the elastic modulus in each condition was obtained by using the material physical property estimation model described above. Among these, trial production was actually performed in the trial production condition with the highest elastic modulus, and the measured value of the elastic modulus was compared with the estimated value. The difference of the estimated value from the measurement value was 6.4%.

For comparison, the learning data including 401 explanatory variables obtained by converting the learning data described above into descriptors was used as it was, and thus a material physical property estimation model was generated. An estimated value of the elastic modulus was obtained by applying the trial production condition with the highest estimated elastic modulus among the estimation conditions described above to the estimation model generated herein in the information processing system according to the embodiment. As a result, the difference of the estimated value from the measured value was 48%, which indicated a greatly lower precision than that of the information processing system of the present embodiment.

As described above, it has been shown that by using the information processing system according to the embodiment, the material physical properties can be successfully estimated with a sufficiently high precision without using a special knowledge even without a lot of learning data.

### OTHER EMBODIMENTS

To be noted, the present disclosure is not limited to the embodiments and examples described above, and can be modified in many ways within the technical concept of the present disclosure. For example, all or part of different embodiments and examples described above may be combined for implementation.

The present disclosure can be also realized by supplying a program realizing one or more functions of the embodiment to a system or an apparatus via a network or a storage medium and performing processing in which one or more processors of a computer of the system or the apparatus read and execute the program. In addition, the present disclosure can be also realized by a circuit (for example, ASIC) that realizes one or more functions.

The operation portion 1604 (FIG. 10) serving as an input portion has a function of receiving input of information (command, data, selection result of options, and the like) required for executing the processing (for example, steps S1 to S12 illustrated in FIG. 9) from the user.

The display apparatus 1605 (FIG. 10) serving as a display portion has a function of displaying information (contents, progress, options, warnings, and the like) related to the executed processing when the information processing unit 103 executes each processing (steps S1 to S12) illustrated in FIG. 9.

According to the present disclosure, an information processing system capable of appropriately identifying important explanatory variables and estimating the material physical properties with high precision even in the case where not a lot of learning data is available can be realized.

### OTHER EMBODIMENTS

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2024-029114, filed Feb. 28, 2024, which is hereby incorporated by reference herein in its entirety.

What is claimed is:

1. An information processing method performed by an information processing unit, the information processing method comprising:
obtaining descriptors related to a material;
converting the obtained descriptors into correlativity-reduced descriptors in which correlativity therebetween is reduced by using conversion coefficients;
calculating importance of the correlativity-reduced descriptors;
identifying important descriptors from the descriptors on a basis of the conversion coefficients used for conversion into the correlativity-reduced descriptors having high importance;
generating a learned model by machine learning using the important descriptors and a characteristic value of the material as learning data; and
estimating a characteristic value of a different material different from the material by using the learned model.

2. The information processing method according to claim 1, wherein in the identifying the important descriptors, a predetermined number of the descriptors are identified as the important descriptors when conversion coefficients used for conversion into the correlativity-reduced descriptors are largest.

3. The information processing method according to claim 1, wherein in the identifying the important descriptors, the descriptors are identified as the important descriptors when conversion coefficients used for conversion into the correlativity-reduced descriptors exceed a predetermined threshold value.

4. The information processing method according to claim 1, wherein in the identifying the important descriptors, the important descriptors are identified based on the conversion coefficients used for conversion into the correlativity-reduced descriptors having high importance.

5. The information processing method according to claim 1, wherein in the identifying the important descriptors, the important descriptors are identified based on weighted averages of the conversion coefficients used for conversion into the correlativity-reduced descriptors, wherein the conversion coefficients being weighted by importance.

6. The information processing method according to claim 1, further comprising displaying information related to processing executed by the information processing unit on a display portion.

7. The information processing method according to claim 1, wherein the information processing unit receives information needed for executing processing via an input portion.

8. The information processing method according to claim 1, wherein the obtained descriptors include a structural descriptor.

9. A non-transitory computer-readable recording medium storing a program for the information processing unit to execute the information processing method according to claim 1.

10. An information processing system comprising an information processing unit,
wherein the information processing unit is configured to:
obtain descriptors related to a material;
convert the obtained descriptors into correlativity-reduced descriptors in which correlativity therebetween is reduced by using conversion coefficients;
calculate importance of the correlativity-reduced descriptors;
identify important descriptors from the descriptors on a basis of the conversion coefficients used for conversion into the correlativity-reduced descriptors having high importance;
generate a learned model by machine learning using the important descriptors and a characteristic value of the material as learning data; and
estimate a characteristic value of a different material different from the material by using the learned model.

11. The information processing system according to claim 10, wherein the information processing unit is configured to identify the important descriptors so as to a predetermined number of the descriptors of which the conversion coefficients used for conversion into the correlativity-reduced descriptors are largest are identified as the important descriptors.

12. The information processing system according to claim 10, wherein the information processing unit is configured to identify the important descriptors so as to the descriptors of which the conversion coefficients used for conversion into the correlativity-reduced descriptors exceed a predetermined threshold value are identified as the important descriptors.

13. The information processing system according to claim 10, wherein the information processing unit is configured to identify the important descriptors so as to the important descriptors are identified on a basis of the conversion coefficients used for conversion into the correlativity-reduced descriptors having high importance.

14. The information processing system according to claim 10, wherein the information processing unit is configured to identify the important descriptors so as to the important descriptors are identified on a basis of weighted averages of the conversion coefficients used for conversion into the correlativity-reduced descriptors, the conversion coefficients being weighted by importance.

15. The information processing system according to claim 10, further comprising a database storing the descriptors related to the material.

16. The information processing system according to claim 10, further comprising a display portion configured to display information related to processing executed by the information processing unit.

17. The information processing system according to claim 10, further comprising an input portion configured to receive information needed for the information processing unit to execute processing.

18. The information processing system according to claim 10, wherein the obtained descriptors include a structural descriptor.
